(19) United States
(12) Patent Application Publication
(10) Pub. No.: US 2024/0302287 A1
(43) Pub. Date: Sep. 12, 2024

(54) WHITE NANOZYME TEST STRIP AND ITS PREPARATION METHOD AND APPLICATION

(71) Applicant: Qingdao Agricultural University, Qingdao (CN)

(72) Inventors: Lei HAN, Qingdao (CN); Yucui ZHANG, Qingdao (CN)

(73) Assignee: Qingdao Agricultural University, Qingdao (CN)

(21) Appl. No.: 18/136,348

(22) Filed: Apr. 19, 2023

(30) Foreign Application Priority Data
Mar. 7, 2023 (CN) 202310209126.9

(51) Int. Cl. G01N 21/78

(52) U.S. Cl. CPC G01N 21/78; G01N 2021/7759

(57) ABSTRACT

A white nanozymes test strip includes a substrate, an organic developer, and a manganese-based metal-organic framework nanozyme is provided, the manganese-based metal-organic framework nanozymes and the organic developer are fixed on the substrate. The present invention uses a white nanozymes test strip as described above, which is inexpensive, simple to prepare and high stability, can resist the interference of oxygen, and the test results are easier to observe, solving the problem that the color development results of colored nanomaterials are not easy to observe, thus improving the detection sensitivity.

### CROSS REFERENCE TO THE RELATED APPLICATIONS

This application is based upon and claims priority to Chinese Patent Application No. 202310209126.9, filed on Mar. 7, 2023, the entire contents of which are incorporated herein by reference.

### TECHNICAL FIELD

The present invention relates to the technical field of test strips, in particular to a white nanozymes test strip and its preparation method and application.

### BACKGROUND

At present, enzyme-based paper analysis devices are widely used in colorimetric, fluorescent and microfluidic analysis due to their simplicity of production, rapid operation and accurate detection. For example, HRP/H2O2/TMB-based paper strips enable realize the colorimetric detection of glucose on paper. However, biological enzymes are expensive, difficult to prepare and have poor stability. As an emerging alternative to enzymes, nanozymes are nanomaterials with enzyme-like activity and are of great interest due to their low cost, ease of preparation, good stability and high catalytic activity. Therefore, nanozymes have broad application prospects for catalyzing color development reactions on paper.

The known nanozyme usually have a variety of colors, such as yellow for vanadium pentoxide (V2O5) nanozyme, black for ferric oxide (Fe3O4) nanozyme and brown for manganese dioxide (MnO2) nanozyme. Although a large dose of nanozymes evenly distributed on paper can amplify the signal and promote activity, its rich color can interfere with the judgement of color development results on paper, thus reducing sensitivity. Technicians often choose to increase the activity of the nanozymes to improve sensitivity, and there are currently few solutions that utilize white nanozymes.

Peroxide-mimetic nanozymes are the most common subfamily of nanozymes and are widely used in colorimetric assays where the nanozymes catalyze the oxidation of the chromogenic substrate with H2O2 to produce a chromogenic product. However, many peroxidase-like nanozymes also have oxidase-like activity and the presence of oxygen can interfere with assays based on peroxidase-like activity. Therefore, it is urgent to explore a kind of nanozymes with only peroxidase-like activity but not oxidase-like activity for colorimetric methods.

Hydrogen peroxide (H2O2) plays a key role in the chemical, food and pharmaceutical industries. However, excessive ingestion of H2O2 will have a number of dangerous effects such as vomiting, mucous membrane irritation and esophagus burns. Therefore, rapid and accurate detection of H2O2 is very important. Moreover, the H2O2 assay is the basis for many assays and can be extended to the detection of multiple targets, such as glucose, sarcosine and pesticides. Therefore, it is important to develop a convenient method for the detection of H2O2. Currently, the detection of hydrogen peroxide based on nanozymes is mostly carried out in solution, and the nanozymes used is colorful. It is urgent to prepare a test strip based on white nanozymes to realize portable and interference-resistant visual and rapid detection of H2O2 on paper.

### SUMMARY

The purpose of the present invention is to provide a white nanozyme test strip, cheap, simple to prepare and high stability, which can resist the interference of oxygen, observe the detection results more easily, and solve the problem that the color development results of colored nanomaterials are not easily observed, thus improving the detection sensitivity. Another object of the present invention is to provide a method for the preparation of white nanozymes test strips and application.

To achieve the above, the present invention provides a white nanozymes test strip, comprising a substrate, an organic developer, and a manganese-based metal-organic framework nanozymes. The manganese-based metal-organic framework nanozymes and organic developer are immobilized on the substrate.

Preferably, the substrate is any one of absorbent paper, cellulose film, or nitrocellulose film.

Preferably, the organic developer is any one of 3,3′,5,5′-tetramethylbenzidine, 2,2′-azino-bis(3-ethylbenzothiazole-6-sulphonic acid) diammonium salt, o-phenylenediamine, 3,3-diaminobenzidine, 4-aminoantipyrine, 5-aminosalicylic acid, 3-amino-9-ethylcarbazole, 4-chloro-1-naphthol.

Preferably, the preparation method of a manganese-based metal-organic framework nanozymes includes the following steps:
(1) Dissolve 0.7-0.8 mmol of terephthalic acid in a mixture containing 30-35 mL of N,N-dimethylformamide (DMF), 1-3 mL of ethanol and 1-3 mL of water;
(2) Add 0.35-0.4 mmol of MnCl2·4H2O to the solution obtained in step (1) under sonication;
(3) Add 0.5-1 mL of triethylamine rapidly to the mixed solution obtained in step (2) and stir for 5-10 min to obtain a white suspension;
(4) The mixed solution obtained in step (3) was sonicated continuously at 30-60 kHz for 8-10 h and centrifuged at 3000-5000 rpm to obtain manganese-based metal-organic framework nanozymes.

The preparation method of above a white nanozymes test strip includes the following steps:

Acetic acid buffer solution (pH 4.0, 100 mmol/L), organic developer and manganese-based metal organic framework nanozymes were evenly applied drops to the surface of substrate and dried at room temperature for 1 h to obtain white nanozymes test strips.

An application of white nanozymes test strips applied to the detection of H2O2 with the following detection steps:

S1. Two test strips, a sample solution containing H2O2 was dropped or soaked on one test strip and a sample solution without H2O2 was dropped or soaked on the other test strip, so that the manganese-based metal-organic framework nanozymes and the organic developer can fully react.

S2. Observe the color change of the test strip to achieve qualitative detection of H2O2, and compare with the test strip without H2O2, if the color turns blue, there is H2O2 in the system.

S3. Take a photo with a smartphone, read the specific RGB value of the test strip in the photo by using the color recognition software of the phone, calculate BN from BN=A [B/(R+G+B)], substitute into the BN-concentration working curve equation to calculate the concentration of H2O2 to achieve quantitative detection of H2O2, where R=Red, G=Green, B=Blue, Δ[B/(R+G+B)]=B/(R+G+B) of strips with H2O2−B/(R+G+B) of strips without H2O2.

The advantages and positive effects of a white nanozymes test strip and its preparation method described in the present invention are as follows:
1. The invention can resist the interference of oxygen, resulting from the fact that the Mn-based metal-organic framework nanozymes has a single peroxidase-like activity and no oxidase-like activity.
2. The results are easier to observe because the Mn-based metal-organic framework nanozymes itself is white, and dropping on the test strips not only achieves whitening effect but also makes the results more visible.
3. The nanozymes is used as catalysts in invention, which is inexpensive, simple preparation and high stability.

The technical solution of the invention is further described in detail below through the accompanying drawings and examples.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIG. 1 is a graph showing the feasibility verification of H2O2 detection on the test strips of the present invention;

FIG. 2 shows a graph of the standard working curve for the quantitative detection of H2O2 of the present invention;

FIG. 3 shows a graph of a standard working curve for the quantitative detection of glucose of the present invention;

FIG. 4 shows a graph for the selectivity assessment of the glucose test strips of the present invention;

FIG. 5 shows a graph of the standard working curve for the quantitative assay for sarcosine of the present invention;

FIG. 6 shows a comparison of the test strips of the present invention before and after the titration of H2O2.

### DETAILED DESCRIPTION OF THE EMBODIMENTS

The technical solutions of the invention are further described below by means of the accompanying drawings and examples.

Unless otherwise defined, technical or scientific terms used in the present invention shall have the ordinary meaning as understood by persons having ordinary skill in the field to which the invention belongs.

### Example 1

Method for the preparation of manganese-based metal-organic framework nanozymes, comprising the steps:
(1) 0.7 mmol of terephthalic acid (BDC) was dissolved in a mixture containing 30 mL of N,N-dimethylformamide (DMF), 1 mL of ethanol and 1 mL of water;
(2) Add 0.35 mmol of MnCl2·4H2O to the solution obtained in step (1) under sonication;
Add 0.5 mL of triethylamine (TEA) to the mixture obtained in step (2) and stir for 5 min to obtain a white suspension;
(4) The mixed solution from step (3) was sonicated continuously at 30 kHz for 8 h and centrifuged at 3000 rpm to obtain a manganese-based metal-organic framework nanozymes.

### Example 2

Method for the preparation of manganese-based metal-organic framework nanozymes, comprising the steps:
(1) Dissolve 0.8 mmol of terephthalic acid (BDC) in a mixture containing 35 mL of N, N-dimethylformamide (DMF), 3 mL of ethanol and 3 mL of water;
(2) Add 0.4 mmol of MnCl2·4H2O to the solution obtained in step (1) under sonication;
(3) Add 1 mL of triethylamine (TEA) rapidly to the mixture obtained in step (2) and stir for 10 min to obtain a white suspension;
(4) The mixed solution from step (3) was sonicated continuously at 60 kHz for 10 h and centrifuged at 5000 rpm to obtain a manganese-based metal-organic framework nanozymes.

### Example 3

A white nanozymes test strip is prepared as follows:

Acetate buffer solution (pH 4.0, 100 mmol/L), 3,3′,5,5′-tetramethylbenzidine (TMB), and manganese-based metal-organic framework nanozymes were evenly applied dropwise to the surface of absorbent paper and dried at room temperature for 1 h to obtain white nanozymes test strips.

### Example 4

A white nanozymes test strip is prepared as follows:

2,2′-Azo-bis(3-ethylbenzothiazole-6-sulfonic acid) diammonium salt (ABTS), manganese-based metal-organic framework nanoparticles, pH 4.0, 100 mmol/L acetate buffer solution, 2,2′-Azo-bis(3-ethylbenzothiazole-6-sulfonic acid) diammonium salt (ABTS) and manganese-based metal-organic framework nanozymes were applied onto the surface of cellulose membrane and dried at room temperature for 1 h to obtain white nanozymes test strips.

### Example 5

A white nanozyme test strip is prepared as follows:

Acetate buffer solution (pH 4.0, 100 mmol/L), o-phenylenediamine (OPD), and manganese-based metal-organic framework nanozymes were applied dropwise to the surface of nitrocellulose membrane and dried at room temperature for 1 h to obtain white nanozymes test strips.

### Example 6

### Feasibility of the Test Strip Test

For the catalytic reaction system (a), test strips were cut up and placed in an acetate buffer solution (100 mmol/L, pH 4.0) containing H2O2 (1 mmol/L) and the reaction solution was taken after 10 minutes for a full wavelength scan. Another control catalytic reaction system (b), test strips were cut up and placed in acetate buffer solution (100 mmol/L, pH 4.0) and the reaction solution was taken after 10 minutes for a full wavelength scan.

As shown in FIG. 1, the catalytic reaction system (a) has a clear UV absorption peak and the catalytic reaction system (b) has no UV absorption peak, proving that the catalytic reaction can only take place in the presence of H2O2.

### Example 7

### Standard Working Curve for the Quantitative Determination of H2O2

(1) Different concentrations of H2O2 (1, 10, 25, 40, 50, 90, 150, 200, 300, 400 μmol/L) were added dropwise to the test strips and left for 1-30 min to allow sufficient interaction of the H2O2 with the manganese-based metal-organic framework nanozymes;
(2) Take a photo with a smartphone, read the specific RGB value of the test strip in the photo by using the color recognition software of the phone, calculate BN from BN=Δ[B/(R+G+B)] to obtain the standard working curve, where R=Red, G=Green, B=Blue, Δ[B/(R+G+B)]=B/(R+G+B) of the test strip with H2O2−B/(R+G+B) of the test strip without H2O2.

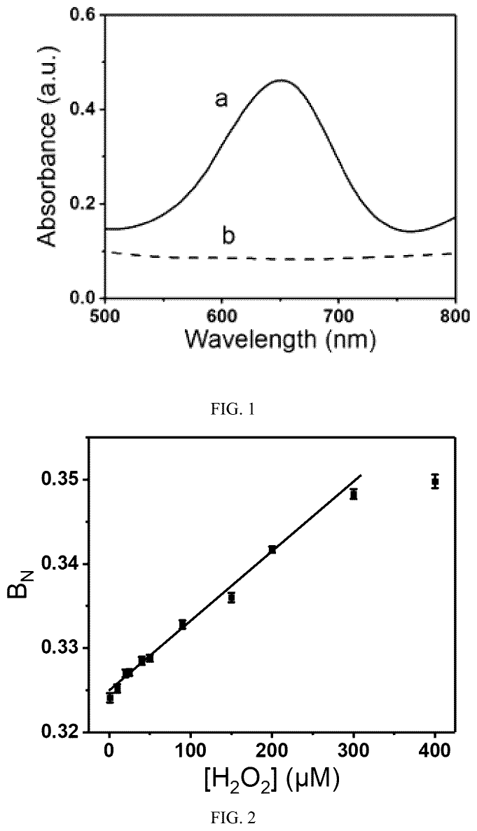

The specific working curve is shown in FIG. 2, with a linear range of 1-300 μmol/L and an equation of y=0.000828208x+0.32485 (R2=0.996).

### Example 8

### Standard Working Curve for the Quantitative Determination of Glucose

(1) Acetate buffer solution (100 mmol/L, pH 4.0), organic developer, manganese-based metal-organic framework nanozymes and glucose oxidase were dripped onto the surface of the substrate and dried at room temperature for 1 h to obtain glucose test strips.
(2) Glucose at different concentrations (0.5, 1, 10, 50, 100, 150, 200, 300, 500, 600 μmol/L) was added dropwise to the test strips and left for 1-30 min to allow sufficient interaction of the glucose, the manganese-based metal-organic framework nanozymes and glucose oxidase.
(3) A smartphone was used to take a photograph and the specific RGB values of the test strips in the photograph were read by using the color recognition software of the phone and the BN was calculated from BN=Δ[B/(R+G+B)] to obtain the standard working curve, where R=Red, G=Green, B=Blue and Δ[B/(R+G+B)]=B/(R+G+B) for the test strip with H2O2−B/(R+G+B) for the test strip without H2O2.

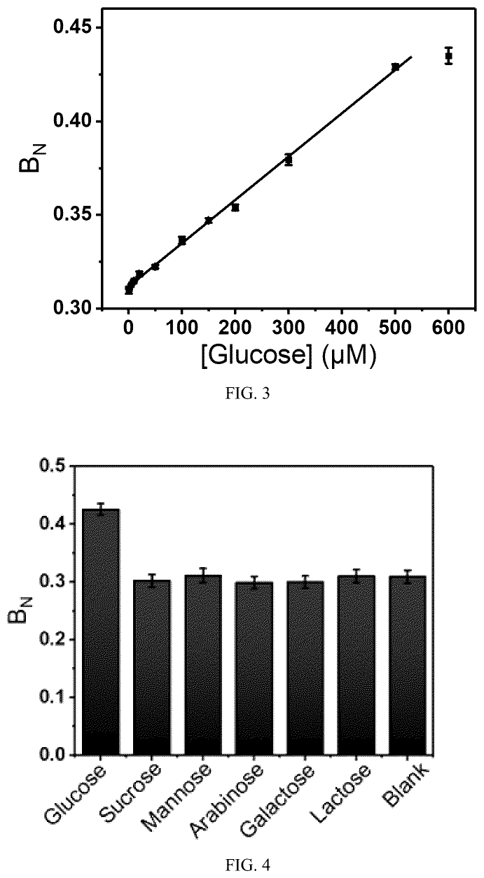

The specific working curve is shown in FIG. 3, with a linear range of 0.5-500 μmol/L and an equation of y=0.000023298x+0.3115 (R2=0.998).

### Selective Assessment of Glucose Detection.

(1) Six test strips with drops of different sugars (glucose, sucrose, mannose, arabinose, galactose, lactose) were taken and left for 1-30 min to allow sufficient interaction of the sugars with manganese-based metal-organic framework nanozymes and glucose oxidase;
(2) Another set of control experiments in which a strip of test paper is taken without a drop of solution and under the same experimental conditions as in step (1);
(3) Take a photograph with a smartphone and read the specific RGB value of the test strip in the photograph by using the color recognition software of the phone to obtain the BN calculated from BN=Δ[B/(R+G+B)] to obtain the selective evaluation chart, where R=Red, G=Green, B=Blue and Δ[B/(R+G+B)]=B/(R+G+B) of the test strip with sugar−B/(R+G+B) of the test strip without sugar.

As shown in FIG. 4, the BN values of the test strips without glucose remained the same as the blank group, which indicated that the glucose test strips were unable to detect the sugars other than glucose as described above, indicating that the test strips were well selective for glucose.

### Example 9

### Standard Working Curve for the Quantitative Determination of Sarcosine

(1) Acetate buffer solution (100 mmol/L, pH 4.0), organic developer, manganese-based metal-organic framework nanozymes and sarcosine oxidase were dripped onto the surface of the substrate and dried at room temperature for 1 h to obtain sarcosine test strips.
(2) The test strips were dripped with different concentrations of sarcosine (2, 5, 15, 25, 50, 100, 150, 200, 300, 400 μmol/L) and left for 1-30 min to allow sufficient interaction of sarcosine, manganese-based metal-organic framework nanozymes and sarcosine oxidase.
(3) Take a photo with smartphone and read the specific RGB value of the test strip in the photo by using color recognition software on phone to obtain a standard working curve by calculating BN from BN=Δ[B/(R+G+B)], where R=Red, G=Green, B=Blue and Δ[B/(R+G+B)]=B/(R+G+B) for the test strip with sarcosine−B/(R+G+B) for the test strip without sarcosine.

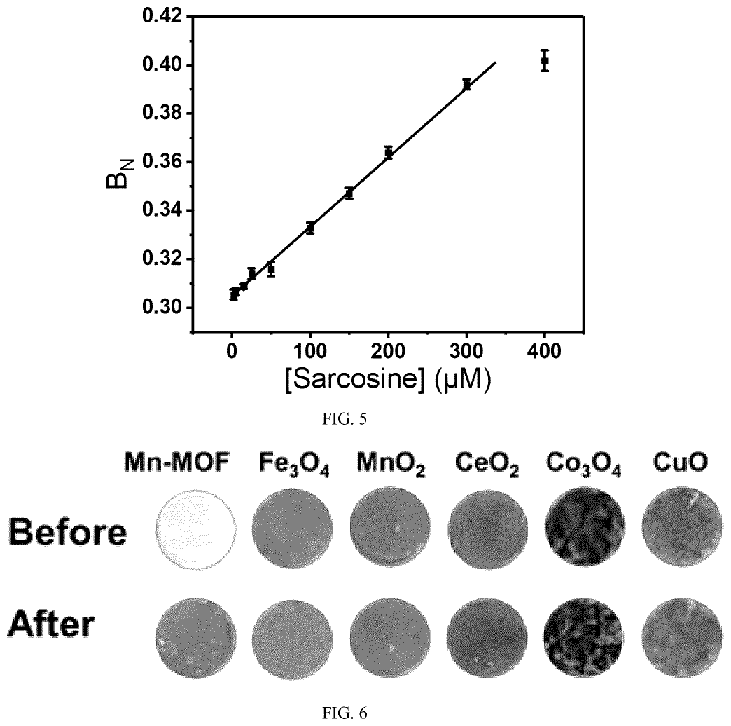

The specific working curve is shown in FIG. 5, with a linear range of 2-300 μmol/L and an equation of y=0.000028823x+0.3044 (R2=0.997).

### Example 10

### Detection of Hydrogen Peroxide in Real Samples.

(1) Sea cucumber and squid were chopped and ground in acetate buffer solution (100 mmol/L, pH 4.0); different concentrations of H2O2 were added to the above extracts respectively, which were centrifuged, filtered through 0.22 μm membrane to remove insoluble impurities and diluted with acetate buffer solution to the appropriate concentration (100 mmol/L, pH 4.0); the above solutions were evenly dropped onto test strips. Then the RGB values identified by phone was substituted into the standard working curve for the quantitative determination of H2O2 to achieve the quantitative determination of H2O2 in the real sample.
(2) The recoveries of the sea cucumber and squid samples were in the range of 99.7-101.9% with the relative standard deviations less than 2.6%.

### Detection of Glucose in Real Samples.

(1) Grind apples and grapes in acetate buffer solution (100 mmol/L, pH 4.0); add different concentrations of glucose to the above extracts respectively, remove insoluble impurities by centrifugation with 0.22 μm membrane filtration, and dilute to the appropriate concentration (100 mmol/L, pH 4.0) with acetate buffer solution; drop the above solutions onto glucose test strips. The RGB values identified by phone was substituted into the standard working curve for the quantitative determination of glucose to achieve the quantitative determination of glucose in the real sample.
(2) The recoveries of the apple and grape samples tested ranged from 98.9 to 100.7% with a relative standard deviation of less than 3.0%.

### Detection of Sarcosine in Real Samples.

(1) Beef and chicken were chopped and ground in acetate buffer solution (100 mmol/L, pH 4.0); different concentrations of sarcosine were added to the above extracts, centrifuged, filtered through 0.22 μm membrane to remove insoluble impurities and diluted with acetate buffer solution to the appropriate concentration (100 mmol/L, pH 4.0); the above solutions were evenly dropped onto sarcosine test strips. The RGB values identified by phone was substituted into the standard working curve for the quantitative determination of sarcosine to achieve the quantitative determination of sarcosine in the real sample.
(2) The recoveries of the beef and chicken samples tested ranged from 99.1-103.2% with a relative standard deviation of less than 3.0%.

### Comparison Example 1

One drop of a sample solution containing H2O2 is added to the test strip, and the other drop of water without H2O2, to allow for a full reaction between the manganese-based metal-organic framework nanozymes and the organic developer.

Observe the color change of the test strip to realize the qualitative detection of H2O2. When the test strip with H2O2 is compared with the strip without H2O2, the color turns blue, indicating that H2O2 was exist in the system.
(1) Acetate buffer solution (pH 4.0, 100 mmol/L), organic developer and colored nanomaterials (Ferric oxide (Fe3O4), Manganese dioxide (MnO2), Cerium dioxide (CeO2), Tricobalt tetroxide (Co3O4) and Copper oxide (CuO)) were applied to the substrate surface in drops and dried at room temperature for 1 h to obtain colored nanozymes test strips.

Add H2O2 dropwise to each of the test strips obtained in step (1) and observe the color change. A separate control experiment was performed in which white manganese-based metal-organic framework (Mn-MOF) nanomaterials were substituted for colored nanomaterials and reacted under the same conditions as the above reaction system and the color change was observed.

As shown in FIG. 6, Before means the test strip without the addition of H2O2, and After refers to the picture of the test strip after the addition of H2O2. The test strips containing Mn-MOF nanozymes show no color without the addition of H2O2, while the blue color of the test strips with the addition of H2O2 is clearly observed. However, the test strips prepared from other colored nanomaterials have no obvious color change before and after the addition of H2O2 due to their own dark color. The use of white nanozymes in the preparation of test strips solves the problem that the color development results of colored nanomaterials are not easy to observe.

### Comparison Example 2

### Preparation of Enzyme Test Strips

(1) First cut out two 3.5 cm diameter circular pieces of test paper and paste them symmetrically onto a 6 cm×10 cm piece of cardboard.
(2) Add 0.27 mL of 3,3′,5,5′-tetramethylbenzidine (TMB) (0.5 mg/mL) solution and 15 μL of horseradish peroxidase (10 μg/mL) solution dropwise to two test strips in a symmetrical distribution and place them in a cold and dry place for 20 min to dry naturally.
(3) Add 15 μL of a sample solution containing a certain concentration of H2O2 dropwise to the horseradish peroxidase-fixed area, pinch the test strip together and separate it after a full reaction for 1 min, and observe the color change of the test strip.

### Procedure for the Preparation of Other Nanozymes Test Strips.

200 μL of 3,3′,5,5′-tetramethylbenzidine (TMB) (5 mmol/L) solution and 100 μL of Pd NPs/meso-C solution (0.33 mg/mL) were added to 4 mL of acetate buffer solution (pH 3.0) and the paper sheets were immersed in the above mixture for 20 s. After removal, the paper sheets were dried in a vacuum oven at 35° C. for 6 h. The paper sheets were dried in a vacuum oven at 35° C. for 6 h.

The white nanozymes test strips of the present invention is prepared by:

Acetate buffer solution (pH 4.0, 100 mmol/L), organic developer and manganese-based metal-organic framework nanozymes were applied dropwise to the substrate surface and dried at room temperature for 1 h to obtain white nanozymes test strips.

Difference: 1. The biological enzyme test strips use pure enzyme horseradish peroxidase as catalyst, which is expensive and has poor stability; the invention uses nanozymes as catalyst, which is inexpensive, simple to prepare and high stability.

2. The catalysts used in other nanozymes test strips are colored nanomaterials, which affect the judgement of the color development results; whereas the present invention uses white nanozymes as the catalyst, which not only whitens the test strips, but also makes the color development results easier to observe, thus improving the detection sensitivity.

Therefore, the present invention uses a white nanozymes test strip as described above, which is inexpensive, simple to prepare and high stability, can resist the interference of oxygen, and the test results are more easily observed, solving the problem that the color development results of colored nanomaterials are not easily observed, thus improving the detection sensitivity.

Finally, it should be noted that the above examples are intended only to illustrate the technical solution of the invention and not to limit it. Despite the detailed description of the invention with reference to the preferred embodiment, it should be understood by those of ordinary skill in the art that modifications or equivalent substitutions can still be made to the technical solution of the invention, and that these modifications or equivalent substitutions do not depart from the spirit and scope of the technical solution of the invention.

1. A white nanozymes test strip comprising a substrate, an organic developer, and a manganese-based metal-organic framework nanozyme, wherein the manganese-based metal-organic framework nanozyme and the organic developer are fixed on the substrate.

2. The white nanozymes test strip according to claim 1, wherein the substrate is any one of an absorbent paper, a cellulose membrane, or a nitrocellulose membrane.

3. The white nanozymes test strip according to claim 1, wherein the organic developer is any one of 3,3′,5,5′-tetramethylbenzidine (TMB), 2,2′-azino-bis(3-ethylbenzothiazole-6-sulphonic acid) diammonium salt (ABTS), o-phenylenediamine (OPD), 3,3-diaminobenzidine (DAB), 4-aminoantipyrine, 5-aminosalicylic acid, 3-amino-9-ethylcarbazole, and 4-chloro-1-naphthol.

4. The white nanozymes test strip according to claim 1, wherein the manganese-based metal-organic framework nanozyme comprises terephthalic acid and MnCl₂·4H₂O.

5. The white nanozymes test strip according to claim 4, wherein the manganese-based metal-organic framework nanozyme is prepared as follows:
(1) dissolving 0.7-0.8 mmol of terephthalic acid in a mixture containing 30-35 mL of N,N-dimethylformamide, 1-3 mL of ethanol, and 1-3 ml of water to obtain a first resulting solution;
(2) adding 0.35-0.4 mmol of MnCl₂·4H₂O to the first resulting solution obtained in step (1) under sonication to obtain a second resulting solution;
(3) adding 0.5-1 mL of triethylamine to the second resulting solution obtained in step (2) rapidly and stirring for 5-10 min to obtain a white suspension; and
(4) sonicating the white suspension obtained from step (3) continuously at 30-60 kHz for 8-10 h and then centrifuging at 3000-5000 rpm to obtain the manganese-based metal-organic framework nanozyme.

6. A method for preparing the white nanozymes test strip according to claim 1, comprising the steps:
dropwise applying a pH 4.0, 100 mmol/L acetate buffer solution, the organic developer, and the manganese-based metal-organic framework nanozyme to a surface of the substrate and drying at room temperature for 1 h to obtain the white nanozymes test strip.

7. A method of using the white nanozymes test strip according to claim 1 in H₂O₂ detection, comprising adding a sample solution containing H₂O₂ to a first white nanozymes test strip.

8. The method of using the white nanozymes test strip in H₂O₂ detection according to claim 7, wherein the detection step is as follows:
S1: taking two of the white nanozymes test strip, dropwise adding the sample solution containing H₂O₂ to the first white nanozymes test strip or soaking the first white nanozymes test strip in the sample solution containing H₂O₂, and dropwise adding a water without H₂O₂ to a second white nanozymes test strip or soaking the second white nanozymes test strip in the water without H₂O₂, allowing a full reaction between the manganese-based metal-organic framework nanozyme and the organic developer;
S2: observing a color change of the two of the white nanozymes test strip to achieve qualitative detection of H₂O₂, comparing the first white nanozymes test strip with H₂O₂ added dropwise to the second white nanozymes test strip without H₂O₂, and if the color becomes blue, then there is H₂O₂ in the first white nanozymes test strip; and
S3: taking a photo with a smartphone, reading a specific RGB value of the two of the white nanozymes test strip in the photo by using a color recognition software of the smartphone, calculating B_{N} from B_{N}=Δ[B/(R+G+B)], substituting into a B_{N}-concentration working curve equation to calculate a concentration of H₂O₂ to achieve a quantitative detection of H₂O₂, wherein R=Red, G=Green, B=Blue, Δ[B/(R+G+B)]=B/(R+G+B) for the first white nanozymes test strip with H₂O₂−B/(R+G+B) for the second white nanozymes test strips without H₂O₂.

9. The method according to claim 6, wherein the substrate is any one of an absorbent paper, a cellulose membrane, or a nitrocellulose membrane.

10. The method according to claim 6, wherein the organic developer is any one of 3,3′,5,5′-tetramethylbenzidine (TMB), 2,2′-azino-bis(3-ethylbenzothiazole-6-sulphonic acid) diammonium salt (ABTS), o-phenylenediamine (OPD), 3,3-diaminobenzidine (DAB), 4-aminoantipyrine, 5-aminosalicylic acid, 3-amino-9-ethylcarbazole, and 4-chloro-1-naphthol.

11. The method according to claim 6, wherein the manganese-based metal-organic framework nanozyme comprises terephthalic acid and MnCl₂+4H₂O.

12. The method according to claim 11, wherein the manganese-based metal-organic framework nanozyme is prepared as follows:
(1) dissolving 0.7-0.8 mmol of terephthalic acid in a mixture containing 30-35 mL of N,N-dimethylformamide, 1-3 mL of ethanol, and 1-3 mL of water to obtain a first resulting solution;
(2) adding 0.35-0.4 mmol of MnCl₂·4H₂O to the first resulting solution obtained in step (1) under sonication to obtain a second resulting solution;
(3) adding 0.5-1 mL of triethylamine to the second resulting solution obtained in step (2) rapidly and stirring for 5-10 min to obtain a white suspension; and
(4) sonicating the white suspension obtained from step (3) continuously at 30-60 kHz for 8-10 h and centrifuging at 3000-5000 rpm to obtain the manganese-based metal-organic framework nanozyme.
